(19) United States
(12) Patent Application Publication
(10) Pub. No.: US 2007/0219577 A1
(43) Pub. Date: Sep. 20, 2007

(54) SPRAYED IN DELIVERY SHEATH TUBES

(75) Inventor: Bradley R. White, Palo Alto, CA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 11/276,996

(22) Filed: Mar. 20, 2006

(51) Int. Cl. A61M 29/00

(52) U.S. Cl. 606/200

(57) ABSTRACT

Embolic protection filtering devices and methods for making and using the same. An example filtering device includes a filter wire and a filter coupled to the filter wire. The filter can include a filter loop and a filter membrane coupled to the filter loop. A plurality of tubular members may be coupled to the filter membrane.

### FIELD OF THE INVENTION

The present invention pertains to embolic protection filtering devices. More particularly, the present invention pertains to embolic protection filtering device with improved delivery features and characteristics.

### BACKGROUND

Heart and vascular disease are major problems in the United States and throughout the world. Conditions such as atherosclerosis result in blood vessels becoming blocked or narrowed. This blockage can result in lack of oxygenation of the heart, which has significant consequences because the heart muscle must be well oxygenated in order to maintain its blood pumping action.

Occluded, stenotic, or narrowed blood vessels may be treated with a number of relatively non-invasive medical procedures including percutaneous transluminal angioplasty (PTA), percutaneous transluminal coronary angioplasty (PTCA), and atherectomy. Angioplasty techniques typically involve the use of a balloon catheter. The balloon catheter is advanced over a guidewire such that the balloon is positioned adjacent a stenotic lesion. The balloon is then inflated and the restriction of the vessel is opened. During an atherectomy procedure, the stenotic lesion may be mechanically cut away from the blood vessel wall using an atherectomy catheter.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

A wide variety of filtering devices have been developed for medical use, for example, intravascular use. Of the known filtering devices, each has certain advantages and disadvantages. There is an ongoing need to provide alternative filtering devices as well as alternative methods for manufacturing filtering devices.

### BRIEF SUMMARY

The invention provides design, material, and manufacturing method alternatives for filtering devices. An example filtering device includes a filter wire and a filter coupled to the filter wire. The filter can include a filter loop and a filter membrane coupled to the filter loop. A plurality of tubular members may be coupled to the filter membrane.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is partial cross-sectional side view of an example filtering device disposed in a blood vessel;

FIG. 2 is a side view of the example filtering device shown in FIG. 1 where the filter is in the collapsed configuration;

FIG. 3 is an alternative side view of the filtering device shown in FIGS. 1-2 along with a filter retaining shaft disposed adjacent the filter;

FIG. 3A is a side view of an alternative filtering device and filter retaining shaft;

FIG. 4 is a side view of the filtering device shown in FIGS. 1-3 where the filter is in the expanded configuration;

FIG. 5 is a side view depicting an example mandrel used in the manufacturing of an example filtering device and the spraying of a filter membrane onto the mandrel;

FIG. 6 is a side view depicting a plurality of tubes disposed on the mandrel and the filter membrane shown in FIG. 5; and

FIG. 7 is a side view illustrating the removal of a portion of the tubes depicted in FIG. 6 from the filter membrane.

### DETAILED DESCRIPTION

The following description should be read with reference to the drawings wherein like reference numerals indicate like elements throughout the several views. The detailed description and drawings illustrate example embodiments of the claimed invention.

When a clinician performs an intravascular intervention such as angioplasty, atherectomy, and the like, embolic debris may dislodge from the blood vessel that can travel in the bloodstream to a position where it may impair blood flow, possibly leading to tissue damage. A number of other situations and/or interventions may also result in the mobilization of embolic debris. Accordingly, embolic protection filtering devices have been developed that can be disposed in the blood vessel downstream of the treatment site and expanded to capture debris.

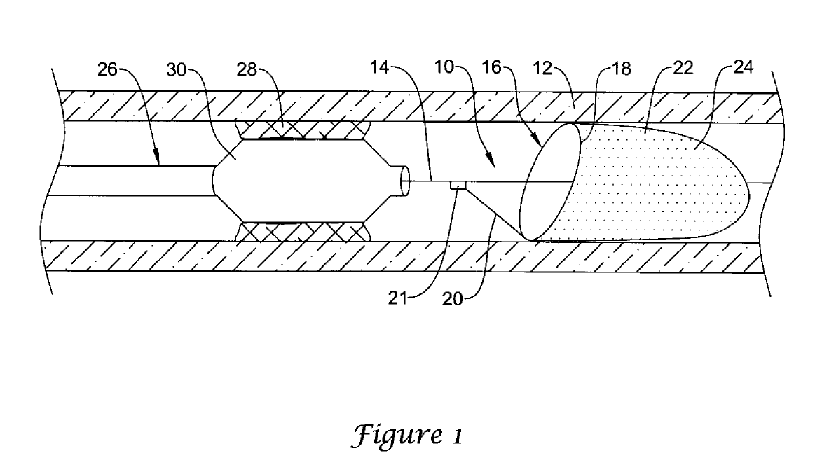

FIG. 1 is a partial cross-sectional view of an example embolic protection filtering device 10 disposed within a blood vessel 12. Device 10 may include an elongate shaft or filter wire 14 having an embolic protection filter 16 coupled thereto. Filter 16 includes a filter loop 18 and a filter membrane or fabric 22 coupled to filter loop 18. Filter membrane 22 can be drilled (for example, formed by known laser techniques) or otherwise manufactured to include a plurality of openings 24. These holes or openings 24 can be sized to allow blood flow therethrough but restrict flow of debris or emboli floating in the body lumen or cavity.

In general, filter 16 may be adapted to operate between a first generally collapsed configuration and a second generally expanded configuration for collecting debris in a body lumen. To this end, in at least some embodiments, loop 18 may be comprised of a “self-expanding” shape-memory material such as nickel-titanium alloy, which is capable of biasing filter 16 toward being in the second expanded configuration. Additionally, filter loop 18 may include a radiopaque material or include, for example, a radiopaque wire disposed about a portion thereof. Some further details regarding these and other suitable materials are provided below.

One or more struts 20 may extend between filter loop 18 and filter wire 14. Strut 20 may be coupled to filter wire 14 by a coupling 21. Coupling 21 may be one or more windings of strut 20 about filter wire 14 or may be a fitting disposed over an end of strut 20 to attach it to filter wire 14. The exact arrangement of struts 20 can vary considerably. One of ordinary skill in the art would be familiar with the various arrangements of struts 20 that are appropriate for a given intervention.

With filter 16 properly positioned in blood vessel 12, another medical device may be advanced over filter wire 14 in order to treat and/or diagnose a lesion 28. For example, a catheter 26 (such as the balloon catheter depicted in FIG. 1) may be advanced over filter wire 14 in order to expand lesion 28. Of course numerous other devices could just as easily be passed over filter wire 14 including any device designed to pass through an opening or body lumen. For example, the device may comprise any type of catheter (e.g., therapeutic, diagnostic, or guide catheter), a stent delivery catheter, an endoscopic device, a laproscopic device, variations and refinements thereof, and the like, or any other suitable device. Alternatively, another device may be advanced over or through its own guiding structure to a suitable location adjacent filter 16 in a manner that allows device 10 to perform its intended filtering function.

Filtering device 10 is generally designed to filter embolic debris that might be generated during the course of this medical intervention. For example, device 10 can be used to capture embolic debris that might be generated during the use of catheter 26 such as when a balloon 30 (coupled to catheter 26) is inflated. It should be noted, however, that device 10 may find utility in concert with essentially any procedure that has the potential to loosen and release embolic debris in to the blood stream or with the devices associated with such procedures.

One characteristic that is often contemplated when designing a filter and/or a filtering invention relates to how the filter is delivered to the proper target location (often termed delivery) and how the filter is removed following the intervention (often termed retrieval). Filtering device 10 includes a number of features that contribute to delivery and/or retrieval. For example, filter frame 16 may be made from a shape-memory material, such as a nickel-titanium alloy, that has the ability to be compressed to a relatively small shape or configuration (e.g., suitable for being disposed in a delivery and/or retrieval catheter) and, when no longer confined, expand to a configuration suitable for filtering within a body lumen.

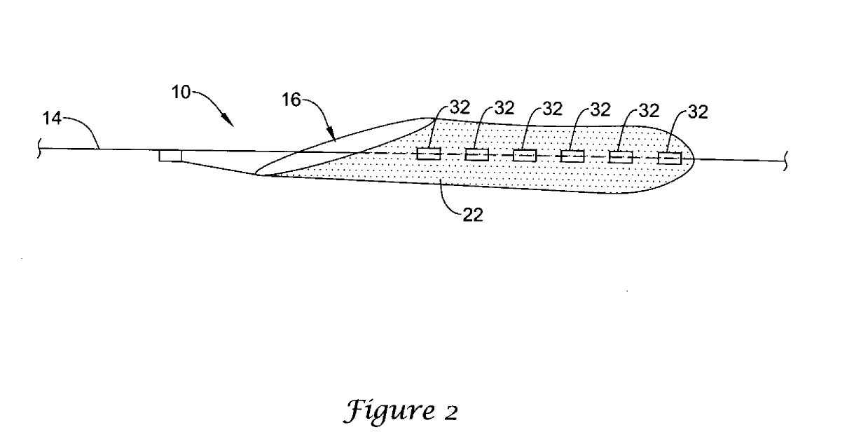

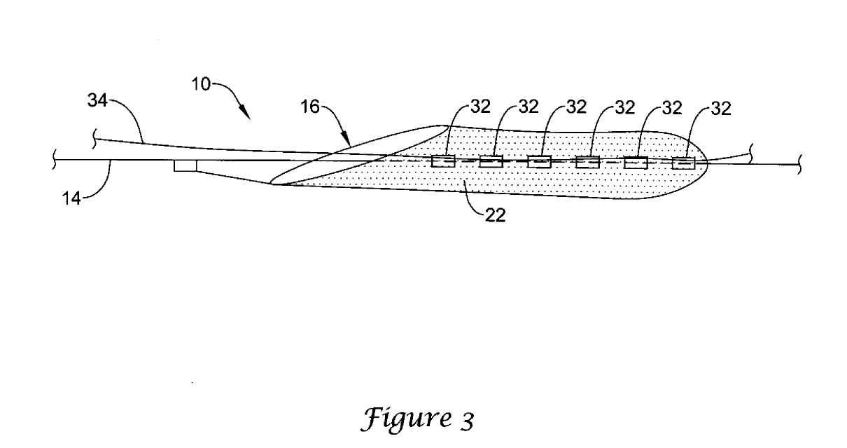

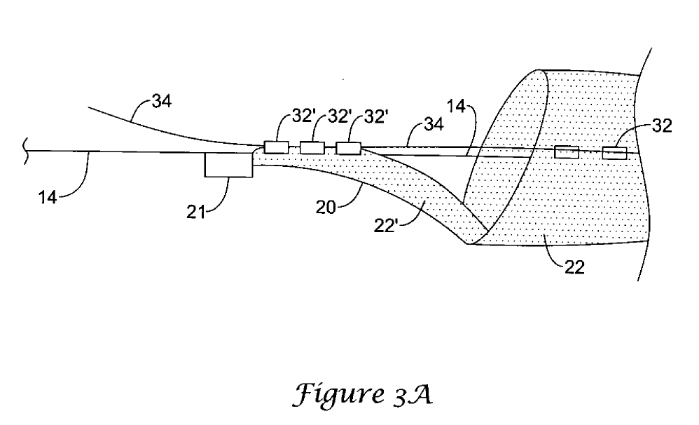

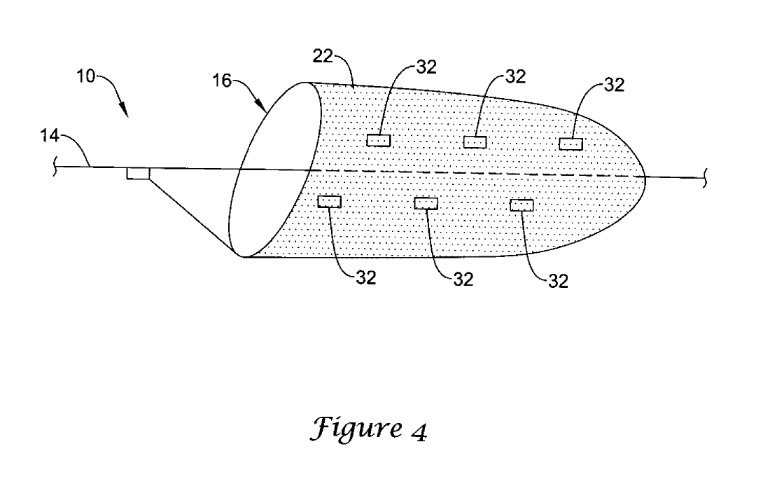

In addition to the aforementioned features, filtering device 10 may also include a number of additional features that relate to delivery and/or retrieval. For example, filtering device 10 may include a plurality of tubular members 32 that are disposed, for example, on filter membrane 22. Turning now to FIG. 2, here it can be seen that tubular members 32 are arranged on the surface of filter 16 (i.e., on the surface of filter membrane 22) so that they longitudinally align when filter 16 is in the collapsed configuration. This allows, for example, a filter retaining shaft 34 to extend through tubular member 32 that substantially holds filter 16 in the collapsed configuration as depicted in FIG. 3. When shaft 34 is removed from tubular members 32, filter 16 shifts to the expanded configuration as depicted in FIG. 4.

In at least some embodiments, an additional segment of filter membrane 22′ (similar to filter membrane 22) can be disposed on strut 20 as shown in FIG. 3A. One or more additional tubular members 32′ (similar to tubular members 32) can be disposed on filter membrane 22′ that can be aligned with each other and/or with tubular members 32 so that filter retaining shaft 34 can extend therethrough. This arrangement may provide additional support for holding filter 16 in the collapsed configuration as well as help retain the position of strut 20 during delivery.

Tubular members 32 can vary considerably in number, arrangement, and form. For example, some embodiments of filtering device 10 include one, two, three, four, five, six, seven, eight, nine, ten, eleven, twelve, thirteen, fourteen, fifteen, sixteen, seventeen, eighteen, nineteen, twenty, or more tubular members 32 disposed on filter membrane 22. Of course, the number of tubular members 32 can vary for different interventions and such variations are contemplated for different embodiments of filtering device 10.

The arrangement of tubular members 32 is generally designed so that at least some of tubular members 32 longitudinally align when filter 16 is collapsed (see FIG. 2). When filter 16 is expanded, at least some of tubular members 32 may become longitudinally misaligned. This is because the collapsing and/or folding of filter 16 changes the shape and surface arrangement of filter 16 so that the longitudinally-aligned positioning of tubular members 32 on a collapsed filter 16 changes to a non-aligned configuration when filter 16 expands.

The pattern need not result in complete misalignment of tubular members 32 when a shift between the collapsed and expanded configuration occurs. For example, some embodiments of filtering device 10 have immediately adjacent tubular members 32 (e.g., a “first” and “second′ tubular member 32) that are longitudinally misaligned when filter 16 is in the expanded configuration. The next adjacent tubular member 32 (e.g., a “third” tubular member) may be longitudinally misaligned with both the other tubular members 32 or longitudinally-aligned with either of the preceding tubular members 32 when filter 16 is expanded. Additional tubular members 32 may be similarly arranged. For example, the additional tubular members 32 may be longitudinally misaligned with some or all of the preceding tubular members 32 and/or longitudinally aligned with some or all of the preceding tubular members 32 when filter 16 is expanded. Such patterns or arrangements of tubular members 32 will vary depending on the amount of folding contemplated for filter 16, the size of filter 16, etc. An additional flap of material could be sprayed onto the support arm with a similar tube arrangement that would help to retain the support arm as well as the body of the filter bag.

Regardless of what arrangement is utilized, at least some of the tubular members 32 are longitudinally-aligned when filter 16 is in the collapsed configuration. This allows filter retaining shaft 34 to be disposed in the longitudinally-aligned tubular members 32 and hold filter 16 in the collapsed configuration. While being held in the collapsed configuration, filter 16 can be more easily delivered and/or retrieved from a target region. Moreover, because filter 16 can be held in the collapsed configuration with shaft 34, the delivery of filtering device 10 can be accomplished without the need of an addition filter delivery sheath or catheter.

Delivery of filtering device 10 may include disposing shaft 34 in tubular members 32 (or otherwise advancing shaft 34 through tubular members 32) so as to hold filter 16 in the collapsed configuration and advancing filtering device 10 through a body lumen (e.g., a blood vessel) to a suitable target region. Once properly positioned, shaft 34 can be proximally retracted, thereby allowing filter 16 to shift to the expanded configuration, suitable for capturing embolic debris. Retrieval of filtering device 10 may include the use of a suitable collapsing structure such as a retrieval sheath (not shown) that can collapse filter 16 to the extent necessary so that shaft 34 can be advanced through at least some tubular members 32 (which may be different tubular members 32 that shaft 34 was disposed in during delivery). With shaft 34 disposed in tubular members 32, the collapsed filter 16 can be removed from the body lumen.

The materials used to manufacture tubular members 32 can vary. In some embodiments, tubular members 32 can be made from metals, metal alloys, polymers, metal-polymer composite, and the like, or any other suitable material. Some examples of suitable metals and metal alloys include stainless steel, such as 304V, 304L, and 316LV stainless steel; mild steel; nickel-titanium alloy such as linear-elastic or super-elastic nitinol, nickel-chromium alloy, nickel-chromium-iron alloy, cobalt alloy, tungsten or tungsten alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si), hastelloy, monel 400, inconel 825, or the like; other Co—Cr alloys; platinum enriched stainless steel; or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

In some embodiments, one or more of tubular members 32 (as well as other portions of filtering device 10 such as filter loop 18 as alluded to above), may also be doped with or otherwise include a radiopaque material. Radiopaque materials are understood to be materials capable of producing a relatively bright image on a fluoroscopy screen or another imaging technique during a medical procedure. This relatively bright image aids the user of filtering device 10 in determining its location. Some examples of radiopaque materials can include, but are not limited to, gold, platinum, molybdenum, palladium, tantalum, tungsten or tungsten alloy, plastic material loaded with a radiopaque filler, and the like.

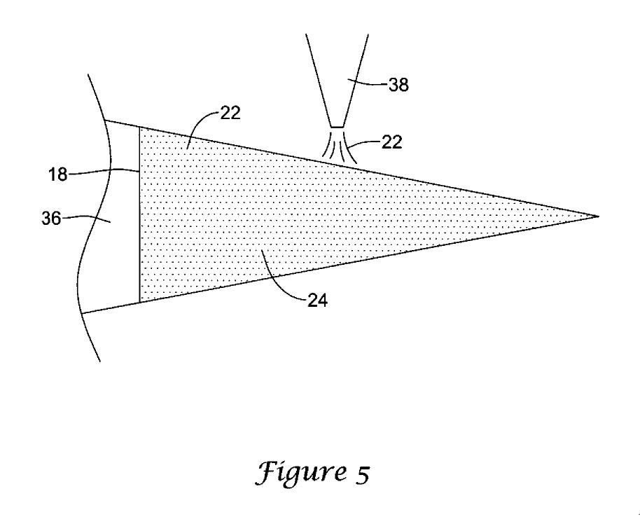

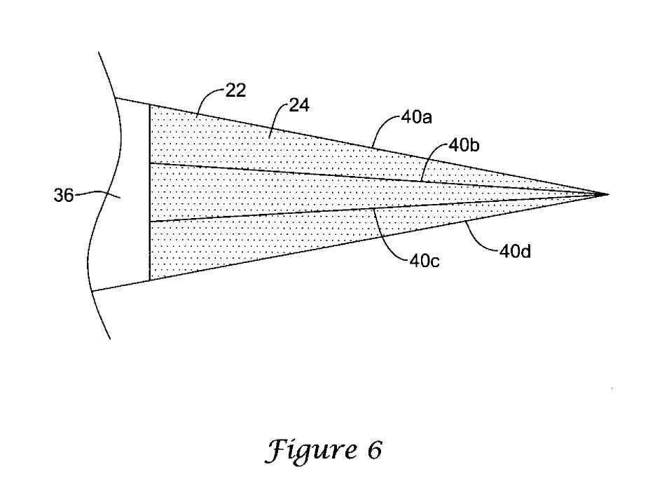

At least some of the steps contemplated for manufacturing filtering device 10 are depicted in FIGS. 5-7. In general, the manufacturing method may include providing a mandrel 36. Mandrel 36 is generally similar to other mandrels used in the filter manufacturing art and has a conical or tapered shape that is characteristic of typical filters. Filter loop 18 can be disposed on mandrel 36 and a spray coating apparatus 38 can be used to spray coat filter membrane 22 onto mandrel 36 and over filter loop 18. With at least a first layer of filter membrane 22 on mandrel 36, one or more tubes 40 (e.g., tubes 40a/40b/40c/40d as shown in FIG. 6) can be disposed on filter membrane 22. In some embodiments, tubes 40 adhere to filter membrane 22. In other embodiments, additional spraying steps are carried out where a second layer of filter membrane 22 is sprayed onto tubes 40. This second spraying step may help secure tubes 40 with filter membrane 22.

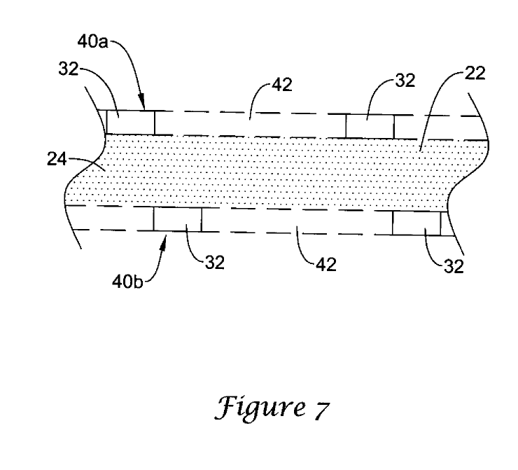

With tubes 40 “sprayed” onto filter material 22, portions of tubes 40 can be removed so as to define tubular members 32. This cutting or removing step is depicted in FIG. 7. Here it can be seen that one or more intermediate portions 42 of tube 40a and tube 40b (as well as however many additional tubes are attached to filter membrane 22) can be removed (as depicted in phantom). The remaining portions of tubes 40a/40b define tubular members 32, which are now attached to filter membrane 22 and can be used as described above. It should be noted that the portions of tubes 40a/40b that are removed (e.g., intermediate portions 42) are generally determined prior to cutting so that the desired arrangement of tubular members 32 when filter 16 is expanded and/or collapsed results from the cutting steps.

In addition to what is described above, the manufacturing method may include any number of additional steps such as forming openings 24 in filter membrane. This may occur in any appropriate manner such as through the use of a laser or any other suitable cutting device.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An embolic protection filtering device, comprising:
an elongate shaft having a distal region;
a filter coupled to the shaft, the filter including a filter loop, one or more struts extending between the filter loop and the shaft, and a filter membrane coupled to the filter loop, the filter membrane having a plurality of openings defined therein; and
a first tubular member coupled to the filter membrane.

2. The filtering device of claim 1, wherein the filter is configured to shift between a first generally expanded configuration and a second collapsed configuration.

3. The filtering device of claim 2, further comprising a second tubular member coupled to the filter membrane.

4. The filtering device of claim 3, wherein the first tubular member and the second tubular member are longitudinally misaligned when the filter is in the expanded configuration.

5. The filtering device of claim 4, wherein the first tubular member and the second tubular member are longitudinally aligned when the filter is in the collapsed configuration.

6. The filtering device of claim 5, further comprising a filter retaining shaft extending through the first tubular member, the second tubular member, and the third tubular member.

7. The filtering device of claim 5, further comprising a third tubular member coupled to the filter membrane.

8. The filtering device of claim 7, wherein the first tubular member and the second tubular member are longitudinally misaligned when the filter is in the expanded configuration and wherein the first tubular member and the third tubular member are longitudinally aligned when the filter is in the expanded configuration.

9. The filtering device of claim 7, wherein the first tubular member, the second tubular member, and the third tubular member are all longitudinally misaligned when the filter is in the expanded configuration.

10. The filtering device of claim 9, wherein the first tubular member, the second tubular member, and the third tubular member are all longitudinally aligned when the filter is in the collapsed configuration.

11. The filtering device of claim 10, further comprising a filter retaining shaft extending through the first tubular member, the second tubular member, and the third tubular member.

12. The filtering device of claim 10, further comprising one or more additional tubular members coupled to the filter membrane.

13. The filtering device of claim 12, wherein all of the one or more additional tubular members are longitudinally misaligned with the first tubular member, the second tubular member, and the third tubular member when the filter is in the expanded configuration.

14. The filtering device of claim 12, wherein at least one of the one or more additional tubular members longitudinally aligns with the first tubular member, the second tubular member, or the third tubular member when the filter is in the expanded configuration.

15. An embolic protection filtering device, comprising:
an elongate shaft having a distal region;
a filter coupled to the shaft, the filter including a filter loop, one or more struts extending between the filter loop and the shaft, and a filter membrane coupled to the filter loop, the filter membrane having a plurality of openings defined therein;
a first tubular member coupled to the filter membrane;
a second tubular member coupled to the filter membrane;
wherein the filter is configured to shift between a first generally expanded configuration and a second collapsed configuration; and
a filter retaining shaft extending through both the first tubular member and the second tubular member to hold the filter in the collapsed configuration, wherein the filter retaining shaft is removable from the first tubular member and the second tubular member, and wherein removing the filter retaining shaft from the first tubular member and the second tubular member shifts the filter to the expanded configuration.

16. The filtering device of claim 15, wherein the first tubular member and the second tubular member are longitudinally aligned when the filter is in the collapsed configuration.

17. The filtering device of claim 15, wherein the first tubular member and the second tubular member are longitudinally misaligned when the filter is in the expanded configuration.

18. The filtering device of claim 15, further comprising one or more additional tubular members coupled to the filter membrane.

19. The filtering device of claim 18, wherein the filter retaining shaft extends through the first tubular member, the second tubular member, and all of the one or more additional tubular members to hold the filter in the collapsed configuration.

20. The filtering device of claim 19, wherein the filter retaining shaft is removable from the first tubular member, the second tubular member, and all of the one or more additional tubular members.

21. The filtering device of claim 20, wherein removing the filter retaining shaft from the first tubular member, the second tubular member, and all of the one or more additional tubular members shifts the filter to the expanded configuration.

22. The filtering device of claim 15, further comprising a second filter membrane coupled to the one or more struts and a third tubular member coupled to the second filter membrane.

23. A method for manufacturing an embolic protection filtering device, comprising the steps of:
providing a mandrel;
disposing a filter loop on the mandrel;
spray coating a first layer of a filter membrane onto the mandrel and over the filter loop;
disposing a first tubular member on the first layer of the filter membrane;
disposing a second tubular member on the first layer of the filter membrane; and
spray coating a second layer of the filter membrane onto the first tubular member and the second tubular member.

24. The method of claim 23, further comprising the step of forming a plurality of openings in the filter membrane.

25. The method of claim 23, further comprising the step of cutting the first tubular member into a plurality of segments.

26. The method of claim 25, further comprising the step of removing at least one of the segments of the first tubular member from the filter membrane.

27. The method of claim 26, further comprising the step of cutting the second tubular member into a plurality of segments.

28. The method of claim 27, further comprising the step of removing at least one of the segments of the second tubular member from the filter membrane.

29. An embolic protection filtering device, comprising:
an elongate shaft having a distal region;
a filter coupled to the shaft, the filter including a filter loop, one or more struts extending between the filter loop and the shaft, and a filter membrane coupled to the filter loop, the filter membrane having a plurality of openings defined therein;
wherein the filter is configured to shift between a first generally expanded configuration and a second collapsed configuration; and
means for holding the filter in the collapsed configuration, wherein at least a portion of the means for holding the filter in the collapsed configuration is attached to the filter membrane.
